(19) United States
(12) Patent Application Publication
(10) Pub. No.: US 2009/0263471 A1
(43) Pub. Date: Oct. 22, 2009

(54) Controlled Release Pharmaceutical Composition of Venlafaxine Hydrochloride, and Process for Preparation Thereof

(76) Inventors: Roberto Valducci, Savignano Sul Rubicone (IT); Tiziano Alighieri, Rimini (IT); Serozh Avanessian, Villa Verucchia (IT)

Correspondence Address: STETINA BRUNDA GARRED & BRUCKER, 75 ENTERPRISE, SUITE 250, ALISO VIEJO, CA, 92656, US

(21) Appl. No.: 11/992,285

(22) PCT Filed: Sep. 29, 2006
(86) PCT No.: PCT/EP2006/066906

§ 371 Date: Mar. 19, 2008

(30) Foreign Application Priority Data
Sep. 30, 2005 (IT) FI2005A000206

(51) Int. Cl. A61K 9/127; A61K 31/137

(52) U.S. Cl. 424/450; 514/649

(57) ABSTRACT

Herein is described a controlled release pharmaceutical composition of venlafaxine hydrochloride, an effective antidepressant, comprising an inert core; on which the active principle is uniformly layered, that in turn is coated with a layer comprising a hardening agent and a lipophilic agent; the process for the preparation of said pharmaceutical composition is also described.

### FIELD OF THE INVENTION

The present invention relates to the field of pharmaceutical compositions, and particularly to a new composition for controlled release of venlafaxine hydrochloride.

### STATE OF THE ART

Venlafaxine, that is 1-[2-dimethylamino-1-(4-methoxyphenyl)-ethyl]cyclohexanol, is a product with recognized activity in the treatment of depression disorders, but also in the treatment of other neurological diseases, such as epilepsy, Parkinson's disease, etc.

The recommended daily dose of the active principle ranges from 75 to 350 mg per day that should be administered in two or three divided doses. Such frequent administrations are particularly complicated, especially considering that patients are affected by neurological diseases. It is therefore evident the need to devise controlled release compositions of venlafaxine or its salts, that allow to avoid frequent drug administration and, at the same time, are suited for the particular pharmacokinetics of venlafaxine salts.

In fact, venlafaxine hydrochloride is a product with high solubility, therefore, if not appropriately formulated; it is released very rapidly, resulting in a high increase of plasma levels of the active principle within a short time after administration, followed by a drop below therapeutic levels approximately 12 hours after administration. Indeed, this makes multiple daily dosage necessary, which causes very undesirable side effects, such as nausea and vomiting in the majority of patients undergoing this dosing regimen.

For the reasons explained above, various controlled release formulations of venlafaxine hydrochloride have been devised to date, wherein the active principle is layered on an inert core and coated with one or more layers of polymeric materials which slow its release. However, it should be said that such formulations are generally prepared by means of complicated and expensive processes, which are not suited for large scale preparation of uniform and stable pharmaceutical compositions.

### SUMMARY OF THE INVENTION

The Applicant has now found that it is possible to modify the release profile of venlafaxine hydrochloride, obtaining a controlled release of the active principle up to 24 hours after administration, by applying a lipophilic coating, as defined below, to the inert cores onto which the active compound has been previously layered.

The active principle so formulated has shown in vitro an ideal type of release in order to achieve in vivo plasma levels suitable for a prolonged pharmaceutical effect, such that a single daily dose administration regimen can be instituted.

Moreover, the present preparation process has shown a good lot-to-lot reproducibility, and a good stability of the compositions obtained.

Therefore, subject of the present invention is a pharmaceutical composition for controlled release of venlafaxine hydrochloride, comprising an inert core on which is applied the active principle venlafaxine hydrochloride, and a lipophilic coating comprising a lipophilic compound and a hardening agent.

The process for preparation of the above-mentioned pharmaceutical composition is a further subject of the invention.

Features and advantages of the invention will be illustrated in detail in the following description.

### SHORT DESCRIPTION OF THE FIGURES

FIG. 1: shows the time profile of the plasma levels of venlafaxine.

FIG. 2: shows the time profile of the plasma levels of O-desmethylvenlafaxine, the main metabolite of venlafaxine.

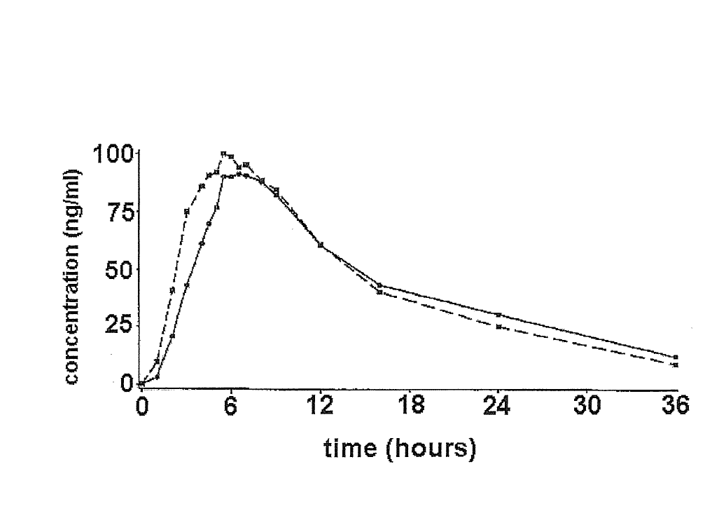

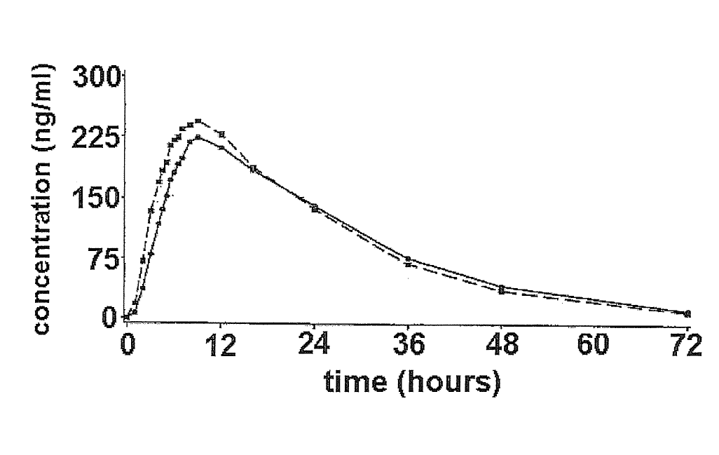

In both FIGS. 1 and 2 the symbol—▪—indicates the curve obtained after administration of one capsule of the invention, prepared as described in Example 2, while the symbol——indicates the reference curve obtained after administration of a venlafaxine hydrochloride sustained release capsule known in the art; and t=0 is the time of administration.

### DETAILED DESCRIPTION OF THE INVENTION

According to the invention, the weight ratio between the lipophilic compound and hardening agent can be comprised, for instance, between 1:1 and 1:20; preferably, the weight ratio between lipophilic compound and hardening agent is 1:10.

According to a preferred embodiment of the invention, the present composition comprises an amount of venlafaxine hydrochloride from 39.3% to 54.0% by weight in respect to the total weight of the composition, and from 1.35% to 2.10% by weight of lipophilic coating in respect to the total weight of the composition; the remaining part of the composition being constituted by the inert core.

Preferred hardening agents are cellulose derivatives, methacrylic acid and its copolymers, polyglycols, polyvinyls, and mixtures thereof.

In the present invention, by the expression “cellulose derivatives” are meant for example products selected from the group consisting of ethylcellulose, methylcellulose, hydroxypropylmethylcellulose, hydroxyethylcellulose, hydroxypropylcellulose, hydroxybutylcellulose, sodium carboxymethylcellulose, hydroxypropylmethylcellulose phthalate, and cellulose acetophthalate; while by the term “polyvinyls” are meant for instance, products chosen among polyvinylacetate, polyvinylpyrrolidone and polyvinylacetophtalate. Polyethyleneglycol is an example of polyglycols according to the invention.

Ethylcellulose is particularly preferred as hardening agent according to the present invention.

Lipophilic compounds of possible use according to the invention are chosen among paraffin, fatty acids with from 12 to 20 carbon atoms, and their mixtures; stearic acid is preferred.

Any inert pellet for pharmaceutical use can be used to prepare the core according to the invention; for instance, pellets made of saccharose and starch can be used. Preferably, the pellets of the invention have a particle size distribution comprised between 710 and 1340 μm.

The present preparation process includes the following steps:
i) application of the active principle on inert pellets by means of nebulization of an aqueous solution of venlafaxine hydrochloride;
ii) application of the lipophilic coating on the core comprising the active principle coming from step i).

The application of the lipophilic compound in mixture with the hardening agent occurs in solution; the organic solvents used for solubilization are selected, for example, from the group consisting of acetone, ethanol, methylene chloride and their mixtures. Mixtures of ethanol and acetone are the preferred solvents for preparation of the solution of lipophilic compound to be applied on the active core. The pharmaceutical compositions according to the invention can comprise, in addition to the above mentioned active principle and components of the lipophilic coating, pharmaceutically acceptable excipients and/or diluents, chosen among those conventionally used for pharmaceutical compositions, in order to realize a composition suitable for controlled release oral administration.

The following examples are provided to illustrate the invention, but are not intended to limit the scope of the present invention.

### Example 1

### Preparation of Active Principle Core

10 Kg Sugar Spheres neutral pellets (composition: 80% by weight saccharose, 20% starch), having particle size distribution between 710 and 1000 μm, are loaded in an automatic system GS model HP/M025.

On these neutral pellets a solution of venlafaxine HCl is applied, which is prepared by solubilization of 10 Kg of active principle in 10 Kg purified water.

The following Table 1 reports the operating conditions for application and the values of the main operating parameters set on the equipment.

TABLE 1

| Parameters | Set-up | |
|---|---|---|
| Nozzle diameter | 1-0.8 | mm |
| Nebulization pressure | 0.5-1.5 | |
| Coater Speed | 16 | rpm |
| Incoming air temperature | 60° | C. |
| Product temperature | 30-40° | C. |

| | |
|---|---|
| Nebulization cycle | continuous |

The so obtained pellets are sieved with a 1200 μm net and dried up for 12 hours at 60° C.

### Example 2

### Preparation of the Controlled Release Coating

290 g of ethylcellulose and 29 g of stearic acid are added to 2.7 Kg of acetone and 2.7 Kg of 96% ethanol, and the mixture is placed under stirring until complete solubilization.

The so obtained solution has been used for application of a retardant film on the pellets prepared as described above in Example 1.

Talc is added in small amounts during nebulization of the solution, for a total amount of 40 g of talc added.

The following Table 2 reports the operating conditions for application of the retardant coat and the values of the main operating parameters set on the equipment.

TABLE 2

| Parameters | Set-up | |
|---|---|---|
| Nozzle diameter | 1.0 | mm |
| Nebulization pressure | 0.5-1.5 | |
| Coater speed | 16 | rpm |
| Incoming air temperature | 60-65° | C. |
| Product temperature | 30-45° | C. |

| | |
|---|---|
| Nebulization cycle | continuous |

The so obtained pellets are sieved with a 1340 μm net and dried up for 12 hours at 60° C. At the end of desiccation, pellets are encapsulated in “0” capsules, comprising 150 mg of venlafaxine corresponding to 169.7 mg of venlafaxine HCl:

### Example 3

### Preparation of the Active Principle Core

2 kg of Sugar Spheres neutral pellets, of the same type already described above in Example 1 are loaded in a Glatt fluid bed model GPCG 3, and a 50% venlafaxine HCl solution is applied on them.

At the end of the application of 4 Kg of solution, pellets are sieved with a 1200 μm net and dried up for 30 minutes at 60° C. in the same fluid bed.

The following Table 3 reports the operating conditions for application and the values of the main operating parameters set on the equipment.

TABLE 3

| Parameters | Set-up | |
|---|---|---|
| Nozzle diameter | 1.2 | mm |
| Incoming air temperature | 35-50° | C. |
| Outgoing air temperature | 22-33° | C. |
| Product temperature | 21-34° | C. |
| Nebulization pressure | 0.5-1.5 | |
| Pump speed | 4-9 | rpm |

### Example 4

### Preparation of the Controlled Release Coating

58 g of ethylcellulose and 5.8 g of stearic acid are added to 0.54 Kg of acetone and 0.54 Kg of 96% ethanol, and the mixture is placed under stirring until complete solubilization.

The so obtained solution has been used for application of a retardant film on the pellets prepared as described above in Example 3 and placed again in fluid bed equipped with Wurster insert.

Talc is added in small amounts during nebulization of the solution, for a total amount of 8 g of talc added.

The following Table 4 reports the operating conditions for application of the retardant coat and the values of the main operating parameters set on the equipment.

TABLE 4

| Parameters | Set-up | |
|---|---|---|
| Nozzle diameter | 1.2 | mm |
| Incoming air temperature | 35-50° | C. |
| Outgoing air temperature | 22-33° | C. |
| Product temperature | 21-34° | C. |

| | |
|---|---|
| Nebulization pressure | 0.5-1.5 |

The so-obtained pellets are sieved with a 1340 μm net and dried up for 12 hours at 60° C. At the end of desiccation, pellets are encapsulated in “0” capsules, containing 150 mg of venlafaxine corresponding to 169.7 mg of venlafaxine HCl.

### Example 5

### Release Test of the Compositions of the Invention

The capsules prepared as described above in Examples 2 and 4 have been analysed by the following method:

Apparatus: Basket 100 rpm; Solubilization medium: H₂O 900 ml

Results of the so performed tests are shown in the following Table 5.

TABLE 5

% release

| Time (hours) | Example 2: | Example 4 |
|---|---|---|
| 2 | 27 | 23 |
| 4 | 48 | 47 |
| 8 | 67 | 69 |
| 24 | 90 | 99 |

### Example 6

### Bioequivalence Test of the Compositions of the Invention

A bioequivalence study has been carried out on the capsules of the invention prepared as in Examples 2 and 4, having as reference the currently marketed sustained release venlafaxine hydrochloride 150 mg capsules.

The results of this study highlighted in FIGS. 1 and 2, show that the product of the invention is equivalent to the product that is currently marketed, both in terms of speed and extent of absorption of the active principle venlafaxine and in terms of speed and extent of formation of its main metabolite O-desmethylvenlafaxine.

1. Controlled release pharmaceutical composition of venlafaxine hydrochloride, comprising an inert core on which is applied the active principle venlafaxine hydrochloride, and a lipophilic coating comprising a lipophilic compound and a hardening agent.

2. Pharmaceutical composition according to claim 1, wherein the weight ratio between lipophilic compound and hardening agent in said lipophilic coating is comprised between 1:1 and 1:20.

3. Pharmaceutical composition according to claim 2, wherein said weight ratio between lipophilic compound and hardening agent is 1:10.

4. Pharmaceutical composition according to claim 1, wherein said hardening agent is selected from the group consisting of cellulose derivatives, methacrylic acid and its copolymers, polyglycols, polyvinyls, and mixtures thereof.

5. Pharmaceutical composition according to claim 1, wherein said hardening agent is selected from the group consisting of ethylcellulose, methylcellulose, hydroxypropylmethylcellulose, hydroxyethylcellulose, hydroxypropylcellulose, hydroxybutylcellulose, sodium carboxymethylcellulose, hydroxypropylmethylcellulose phthalate, cellulose acetophthalate, polyvinylacetate, polyvinylpyrrolidone, polyvinylacetophtalate, polyethyleneglycol and mixtures thereof.

6. Pharmaceutical composition according to claim 5, wherein said hardening agent is ethylcellulose.

7. Pharmaceutical composition according to claim 1, wherein said lipophilic compound is chosen among paraffin, fatty acids with from 12 to 20 carbon atoms, and mixtures thereof.

8. Pharmaceutical composition according to claim 7, wherein said lipophilic compound is stearic acid.

9. Pharmaceutical composition according to claim 1, wherein said inert core essentially consists of neutral pellets composed of saccharose and starch, having a particle size distribution comprised between 710 and 1340 μm.

10. Pharmaceutical composition according to claim 1, further comprising pharmaceutically acceptable excipients and/or diluents.

11. Pharmaceutical composition according to claim 1, in the form of capsule.

12. Pharmaceutical composition according to claim 1, comprising an amount of venlafaxine hydrochloride from 39.3% to 54.0% by weight with respect to the total weight of the composition, and from 1.35% to 2.10% by weight of lipophilic coating with respect to the total weight of the composition; the remaining part of the composition being constituted by the inert core and possibly by pharmaceutically acceptable excipients and/or diluents.

13. Pharmaceutical composition according to claim 12, having the following composition expressed in percent by weight with respect to the total weight of the composition:

| | |
|---|---|
| venlafaxine hydrochloride | 49.10% |
| neutral pellets | 49.16% |
| ethylcellulose | 1.40% |
| stearic acid | 0.14% |
| talc | 0.20% |

14. Process for preparation of the pharmaceutical composition as defined in claim 1, comprising the following steps:
i) application of the active principle venlafaxine hydrochloride on the inert core by means of nebulization of an aqueous solution of venlafaxine hydrochloride;
ii) application on the core comprising the active principle, coming from step i), of the lipophilic coating comprising a lipophilic compound and a hardening agent.

15. Process according to claim 14, wherein application of the lipophilic coating in step ii) is carried out by means of nebulization on the core coming from step i), of a solution comprising the lipophilic compound and the hardening agent in a suitable solvent.

16. Process according to claim 15, wherein said solvent is an organic solvent selected from the group consisting of acetone, ethanol, methylene chloride and mixtures thereof.

17. Process according to claim 16, wherein said organic solvent is a mixture of ethanol and acetone in a 1:1 weight ratio.

18. Process according to claim 15, wherein talc is added during said nebulization of the solution comprising the lipophilic compound and the hardening agent.
